(19) United States
(12) Patent Application Publication
(10) Pub. No.: US 2023/0235289 A1
(43) Pub. Date: Jul. 27, 2023

(54) METHOD FOR FIBROBLAST REJUVENATION BY MECHANICAL REPROGRAMMING AND REDIFFERENTIATION

(71) Applicant: Paul Scherrer Institut, Villigen PSI (CH)

(72) Inventors: G.V. Shivashankar, Zürich (CH); Bibhas Roy, Wettingen (CH)

(21) Appl. No.: 17/918,981

(22) PCT Filed: Mar. 30, 2021
(86) PCT No.: PCT/EP2021/058258

§ 371 Date: Oct. 14, 2022

(30) Foreign Application Priority Data
Apr. 14, 2020 (EP) 20169459.3

(51) Int. Cl. C12N 5/077

(52) U.S. Cl. CPC C12N 5/0656; C12N 2506/1307; C12N 2527/00; C12N 2533/52; C12N 2533/54; C12N 2535/10

(57) ABSTRACT

Over the course of an aging process fibroblasts lose contractility, leading to reduced connective tissue stiffness. A promising therapeutic avenue for functional rejuvenation of connective tissue is reprogrammed fibroblast replacements with a laterally confined growth of fibroblasts on micro-patterned substrates that induces stem cell-like spheroids. The partially reprogrammed spheroids are embedded in collagen-I matrices of varying densities, mimicking different 3D tissue constraints. The spheroids regain their fibroblastic properties and sprout to form 3D connective tissue networks. The differentiated fibroblasts exhibit reduced DNA damage, enhanced cytoskeletal gene expression and acto-myosin contractility. The rejuvenated fibroblasts show increased matrix protein (fibronectin and laminin) deposition and collagen remodeling compared to the parental fibroblast tissue network. The partially reprogrammed cells have comparatively open chromatin compaction states and may be more poised to redifferentiation into contractile fibroblasts in 3D-collagen matrix. Collectively, the results highlight efficient fibroblast rejuvenation, with important implications in regenerative medicine.

The present invention relates to a method for the rejuvenation and the re-differentiation of fibroblast cells.

Fibroblasts are vital constituents of the connective tissue, which provide mechanical strength and maintain tissue homeostasis by promoting extracellular matrix remodeling. During aging, fibroblasts reduce their actomyosin contractility and matrix remodeling efficiencies. Transplanting of stem cell and induced pluripotent stem cells (iPSCs) are being seen as potential cellular therapy model for rejuvenating fibroblast function. However, these interventions not only rejuvenate, but have been found to acquire genomic mutations that may increase the oncogenic potential of the proliferative fibroblasts and major efforts are underway to improve the limitations of such methods. Therefore, for therapeutic purposes, it would also be ideal to rejuvenate fibroblasts using non genetic methods.

Therefore, it is the objective of the present invention to provide a method for the rejuvenation and the re-differentiation of fibroblast cells that create fibroblast cells of high DNA fidelity and enhanced cytoskeletal gene expression and acto-myosin contractility.

This objective is achieved according to the present invention by a method for fibroblast rejuvenation by mechanical reprogramming and redifferentiation, comprising the steps of:

a) laterally confined growing of fibroblasts on micro-patterned substrates in order to induce the generation of stem-cell like spheroids; and
b) embedding these partially reprogrammed spheroids in 3D matrices, preferably Collagen-I matrices, of varying densities, thereby mimicking different 3D tissue constraints.

Thus, the sustained laterally confined growth of fibroblasts on micropatterned substrates induces their reprogramming into stem cell-like cells, even in the absence of any genetic or biochemical interventions. Such partially reprogrammed cells not only exhibited stem cell like characteristics but also retain their differentiation states to some extent, making them a model for fibroblast rejuvenation in connective tissues. As a major constituent of connective tissue, Collagen-I concentration primarily regulates the matrix stiffness and controls the cellular process such as contraction, adhesion, and migration via its interaction with fibroblasts. Furthermore, in a three-dimensional gel, matrix fibers are intertwined into a mesh-like structure and the porosity of the mesh regulates initial cell spreading and migration through the entangled fibrils. Therefore, 3D collagen matrices with appropriate steric (porosity) and mechanical (stiffness) features that closely resemble fibrous connective tissue are ideal for exploring the fate of reprogrammed cells in a tissue-like microenvironment. The present invention therefore discloses the mechanical reprogramming of fibroblasts, followed by their redifferentiation into rejuvenated fibroblasts in an optimized 3D collagen matrix made these cells more contractile and more efficient at synthesizing matrix components including laminin, fibronectin, collagen-IV. Moreover, the rejuvenated fibroblasts obtained through this approach exhibited a decrease in DNA damage. The rejuvenated fibroblasts derived from this method precisely align into tissue architectures, suggesting its potential application as clinical implants in tissue engineering and regenerative medicine.

Preferred embodiments of the present invention are given below and can used alone or in combination:

a) the micro-patterned substrate is a fibronectin micropatterns; preferably a rectangular micropattern having an aspect ratio in the range of 1:5 and measuring in the range of 400 to 3000 μm², preferably around 1,800 μm², are created on uncoated Ibidi dishes by stamping of fibronectin coated PDMS micropillars fabricated by soft lithography;
b) the micropatterned substrate is surface passivated with pluronic acid and fibroblast cells are expanded in high-glucose DMEM and FBS and penicillin-streptomycin;
c) for partial reprogramming, the fibroblast cells are seeded on a fibronectin-micropatterned dish, preferably rectangles spaced in the range of 50 to 250 μm, preferably 150 μm, apart, at a concentration of 2,000 to 20.000 cells per dish, preferably around ˜7,000 cells per dish, to reach a density of one cell per fibronectin island; single cells are grown in under laterally confined conditions for a predetermined amount of time, preferably a couple of days, in the above-mentioned culture medium, preferably with a fresh media replenishment on every alternate day;
d) for a partial reprograming of human fibroblasts (BJ cells), the human fibroblast cells are grown on laterally confined condition on a specific fibronectin micropattern, preferably having an area around 1.000 to 10.000 μm², i.e. around 3364 um² at an aspect ratio of 1:4, for a couple of days in high-glucose DMEM and FBS and penicillin-streptomycin and for the re-differentiation, these partially reprogrammed human fibroblast cells are further re-differentiated by embedding them on a collagen matrix;
e) micro-patterned substrates for partially reprogramming fibroblasts with high efficiency are used;
f) the step of partial reprogramming is executed with patient specific old fibroblasts;
g) a 3D gel protocol to encapsulate reprogrammed old fibroblasts for their rejuvenation is established; and/or
h) patient specific rejuvenated fibroblasts are characterized for potential applications.

Preferred embodiments of the present invention are hereinafter described in more detail with reference to the attached drawings which depicts in:

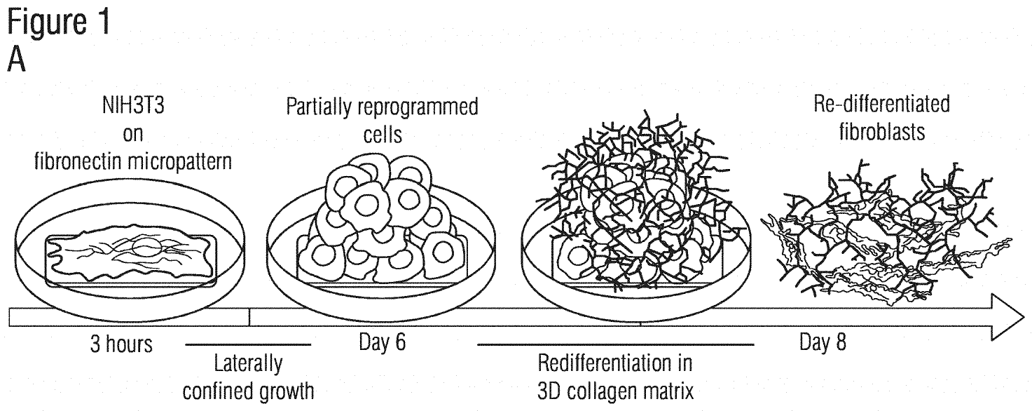

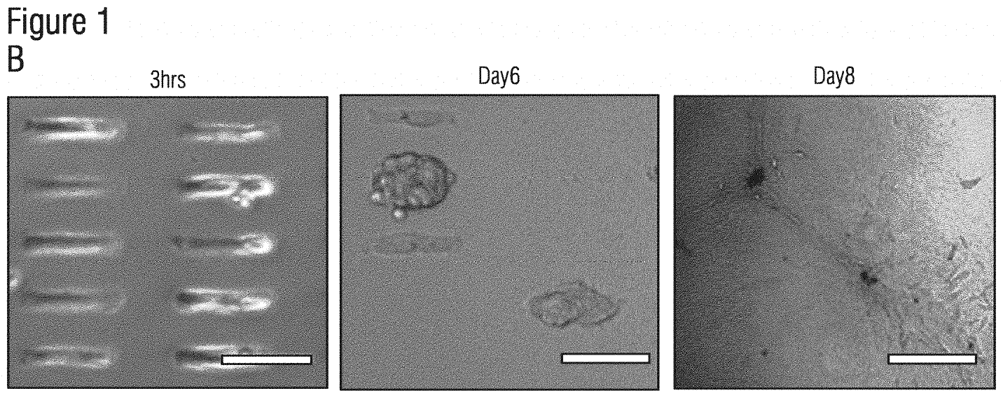

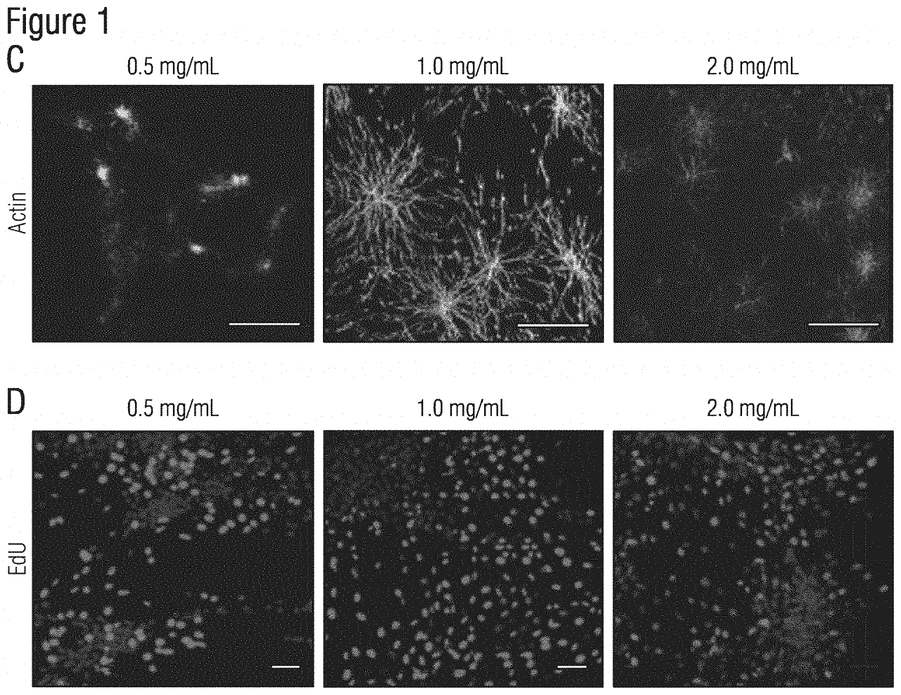

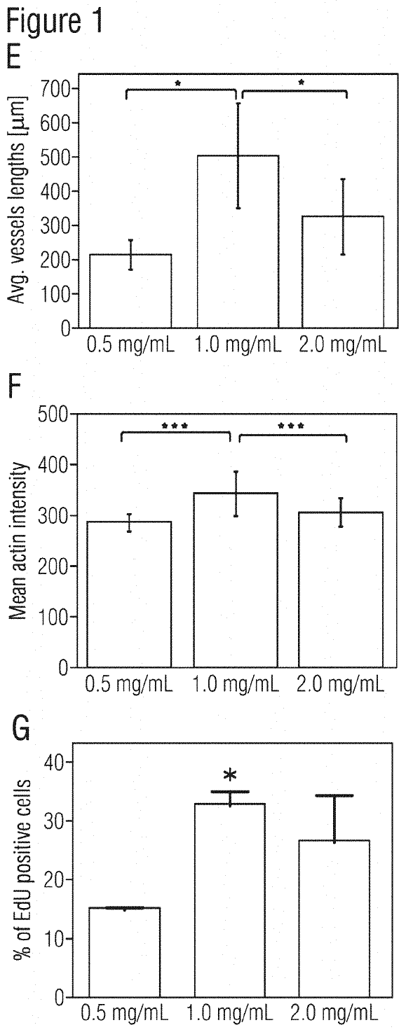

FIG. 1 redifferentiation of fibroblasts from partially reprogrammed spheroids depends on the 3D collagen matrix density; (A) Schematic representation of the effect of geometry-driven laterally confined growth of fibroblasts on reprogramming, followed by their redifferentiation within the embedded 3D collagen matrix. (B) Phase contrast images of NIH 3T3 mouse fibroblast cells cultured on micropatterns for up to 6 days, spheroids of partially reprogrammed cells, and re-differentiated fibroblasts undergoing sprouting in 3D collagen matrix (Scale bar, 100 μm). (C) Sprouting efficiencies of cells from 6 day-old spheroids on collagen matrices of varying densities. Cells are stained with phalloidin to label actin; scale bar: 500 μm. (D) Proliferation rates of sprouting cells at varying matrix density using an EdU incorporation assay; scale bar: 50 μm. (E-G)

Sprouting efficiencies, contractility, and proliferation levels at varying matrix density measured by average vessel length, mean actin intensities, and percentages of EdU-positive cells in each field of view, respectively. For FIGS. 1E and G, n>5 fields of view were randomly measured in each condition. For FIG. 1F, number of cells, n=3304, 4786 and 1015 for 0.5, 1 and 2 mg/mL condition, respectively. Error bars represent±SD; *P<0.05; **P<0.01; ***P<0.001; Two-sided Student's t-test was used.

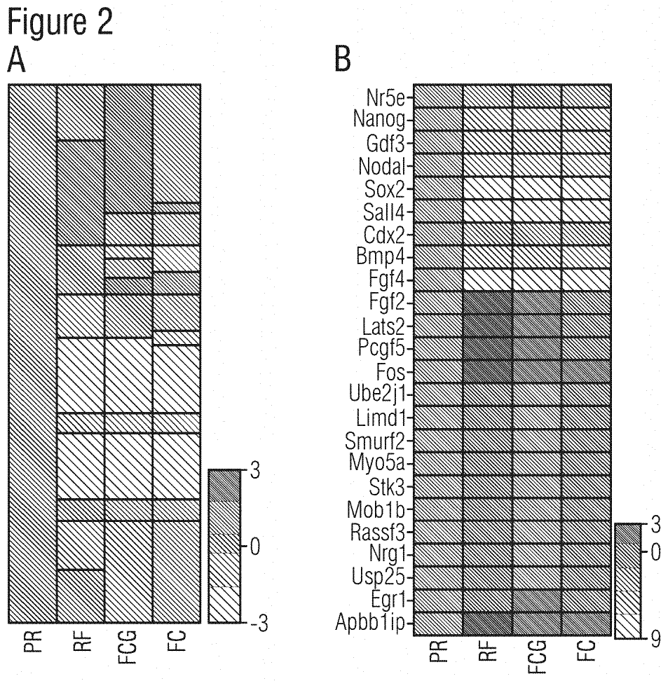

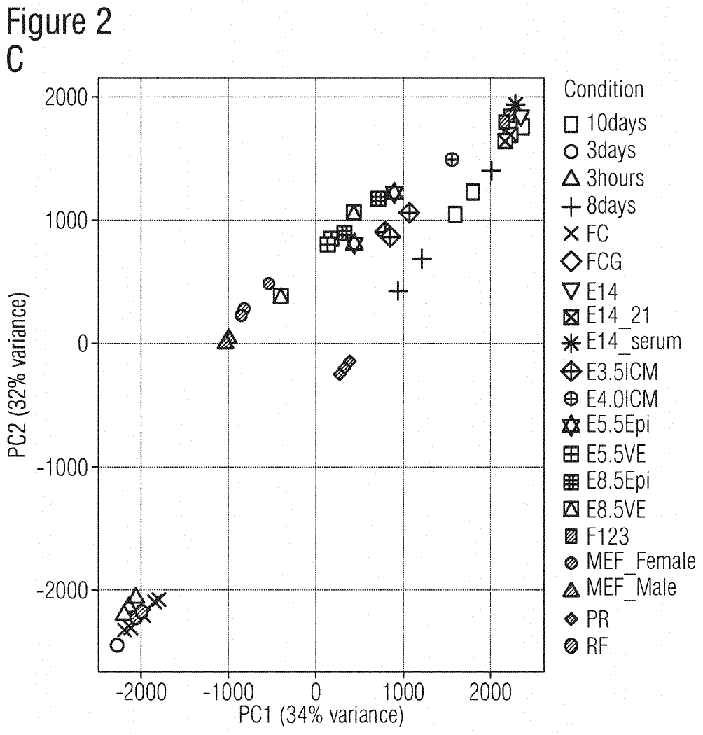

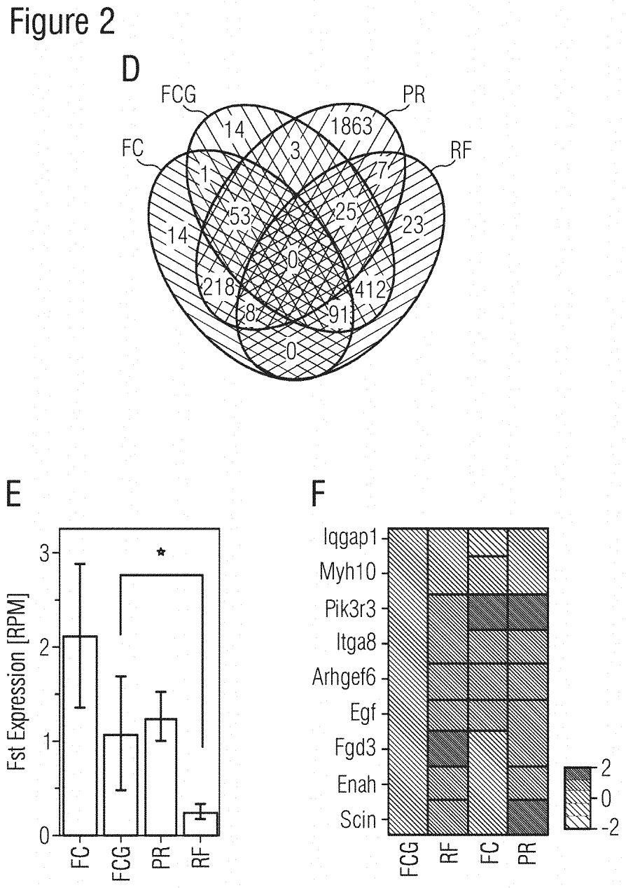

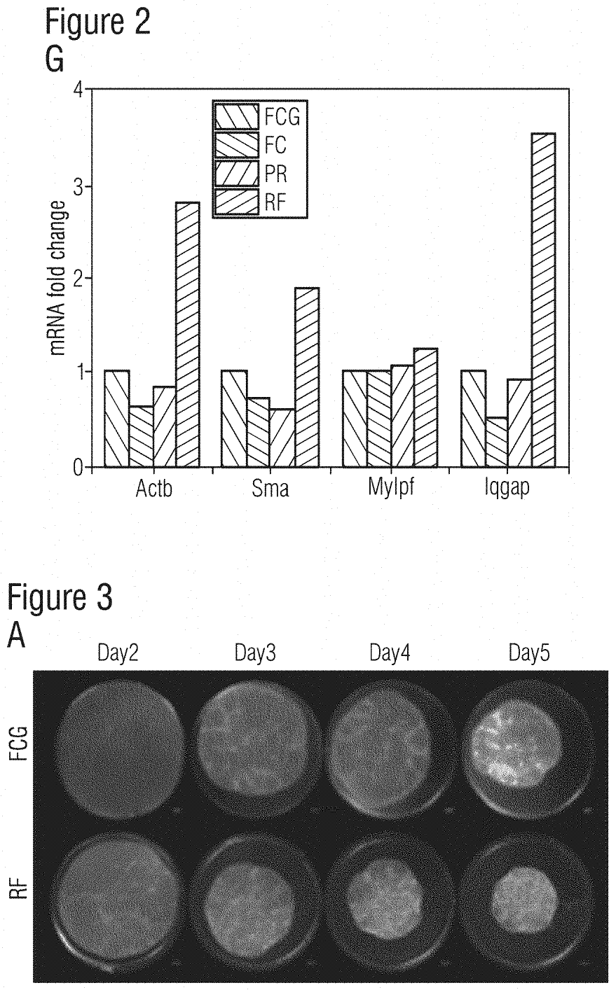

FIG. 2 a shift in transcription profile accompanied with enhanced cytoskeletal genes' expression. (A) Heatmap showing fold change (log2) in global transcription profiles between PR, RF, FCG and FC cells. FDR (adjusted p value)<0.1. (B) Heatmap showing log2 fold change in the differential Day 6 partially expression of selected genes (compared to the PR sample). FDR (adjusted p value)<0.1. (C) Principle component analysis (PCA) showing the shift in cell states through reprogramming and reverting back through redifferentiation. FDR (adjusted p value)<0.01 and |log2 Fold change|>2. (D) Venn diagram showing the number of upregulated genes in 14 (2ⁿ-2, n is 4 conditions) comparisons. FDR (adjusted p value)<0.1. (E) Bar plot depicting changes in the expression of the representative aging gene Follistatin (Fst). The error bars represent±SD; *P<0.1. (F) Heatmap showing the log2 fold change in the differential expression of selected cytoskeletal-related genes (compared to FCG). P value (not adjusted)<0.05. (G) mRNA levels of selected cytoskeletal genes obtained by qRT-PCR. Normalized with respect to FCG.

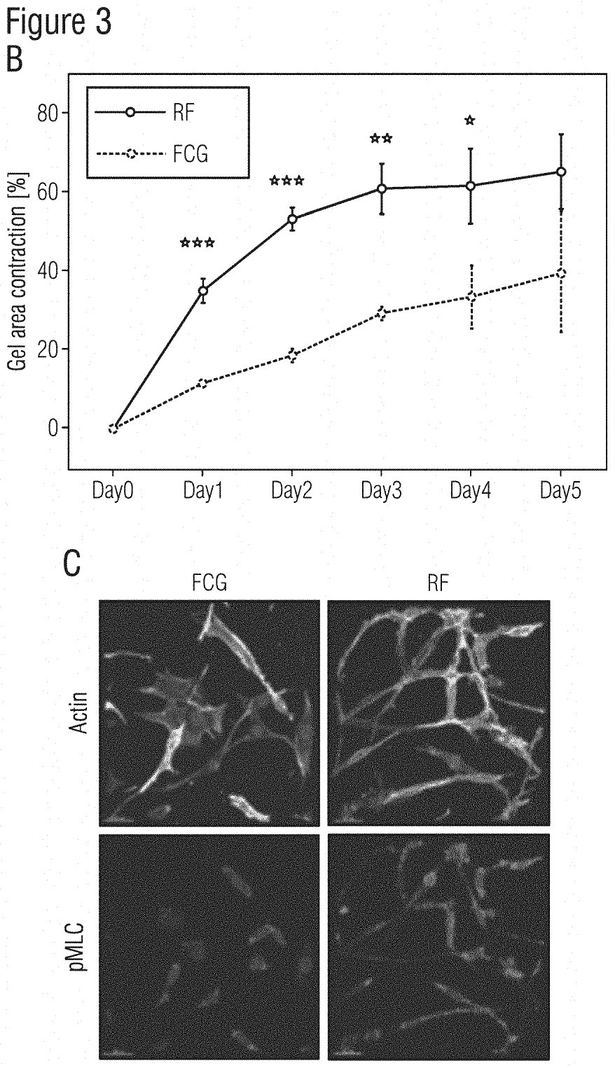

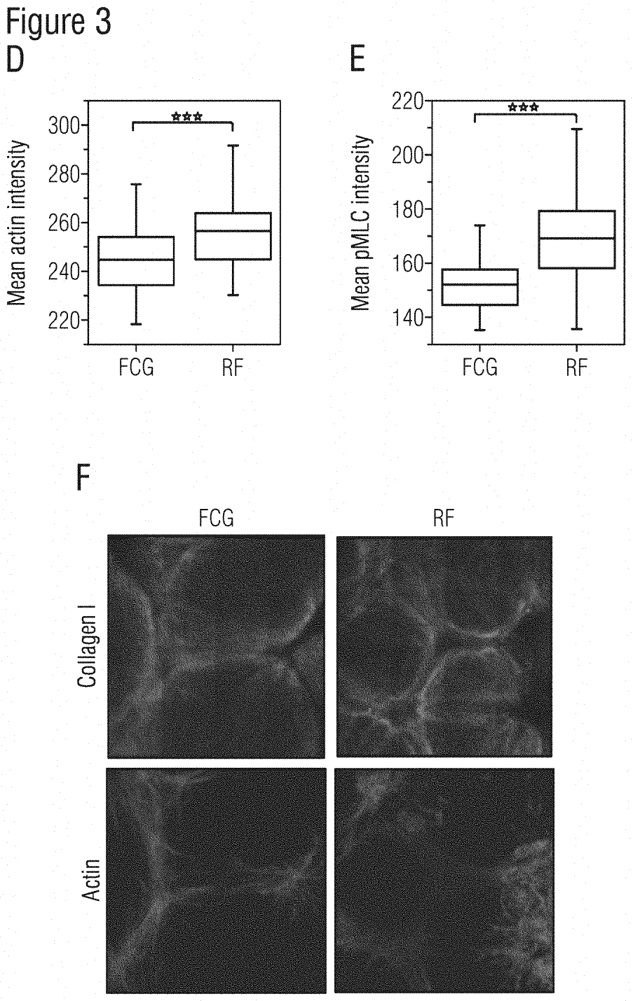

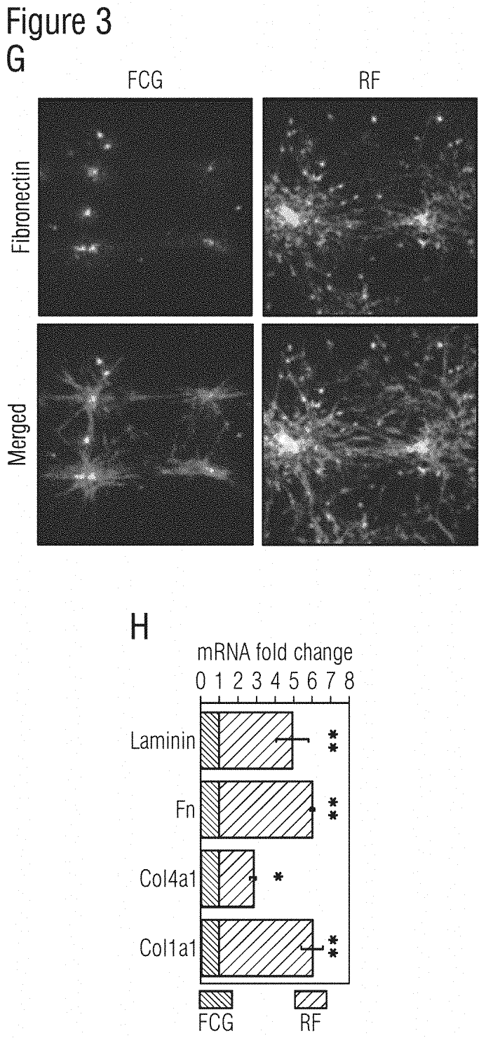

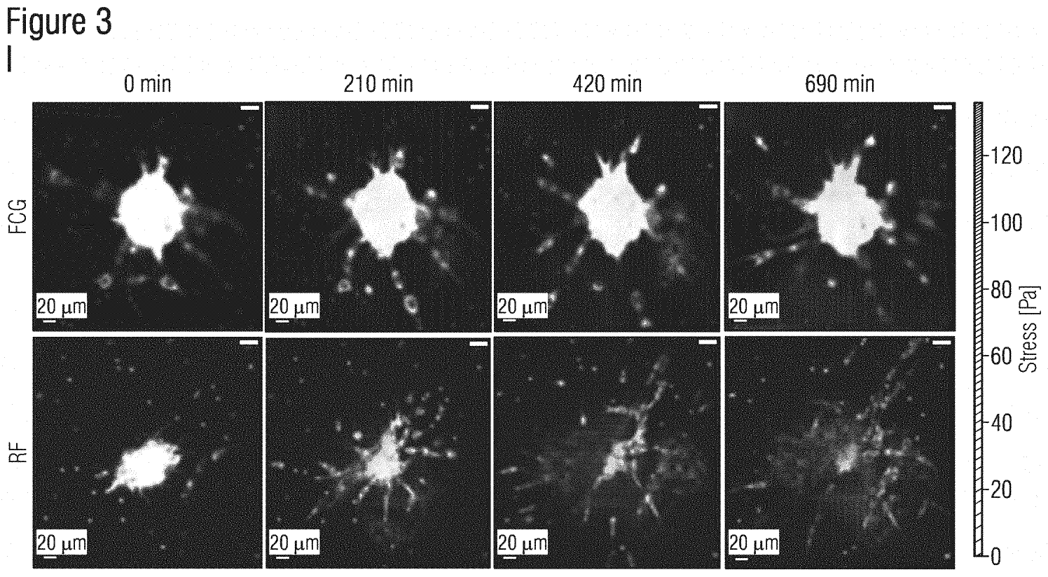

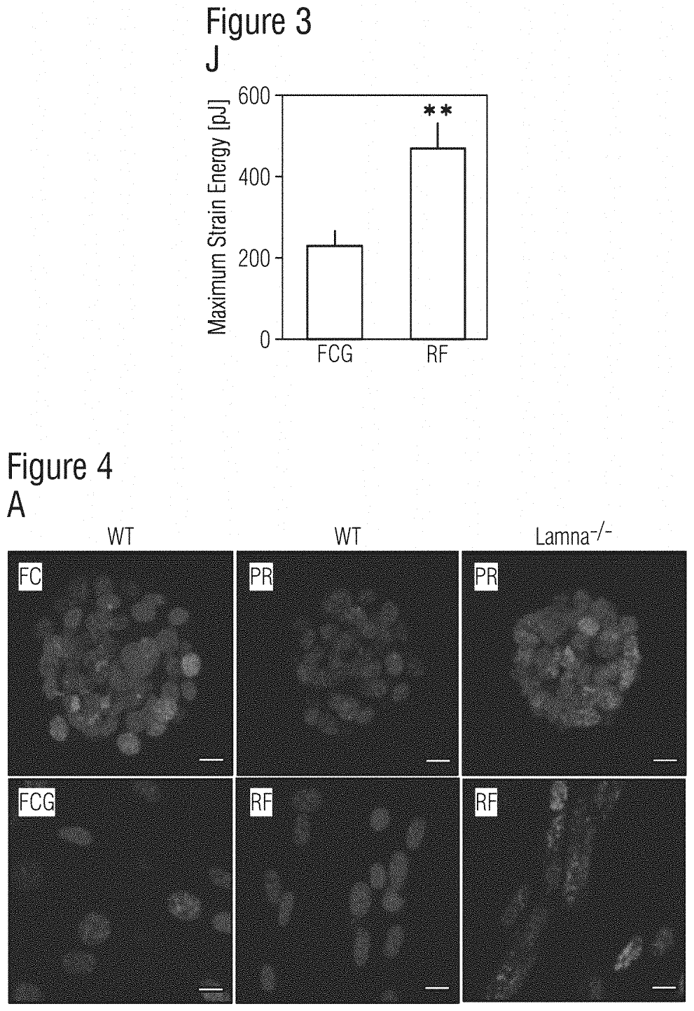

FIG. 3 re-differentiated Fibroblasts are characterized by enhanced contractility and matrix remodeling. (A) Representative images of temporal collagen gel contraction by matrix-embedded, RF and FCG. (B) Normalized gel area plot representing the gel contraction efficiencies of these two types of cells. Error bars represent±SD. The p-values represent the adjusted p-values obtained by Bonferroni adjustment methods. *P<0.05; **P<0.01; ***P<0.001, Two-sided Student's t-test was used. (C) Representative actin and pMLC immunofluorescence micrograph of RF and FCG embedded in 1 mg/ml collagen matrix; scale bar: 20 μm. (D and E) Corresponding box plots for cellular mean intensity of actin and pMLC; n=81 and 67 for FCG and RF conditions, respectively. ***P<0.001; two-sided Student's t-test were used. (F) Representative fluorescence micrographs of immunostained collagen matrix in these two conditions. Corresponding phalloidin-stained actin images represents cells within the matrix. (G) Representative immunofluorescence micrographs of extracellular fibronectin deposited on to the matrix in these two conditions. In merged images, the nucleus is labeled in blue, fibronectin in green and actin in red. (H) mRNA levels of selected extracellular matrix-related genes obtained by qRT-PCR. Normalized with respect to FCG. Error bars represent±SD *P<0.05; **P<0.01; Two-sided Student's t-test was used. (I) Representative temporal traction force maps quantifying forces exerted on the matrix during sprouting of these two cell types. (J) Corresponding maximum strain energy plots during sprouting of these two cell types. Error bars represent±SD; **P<0.01; Two-sided Student's t-test was used.

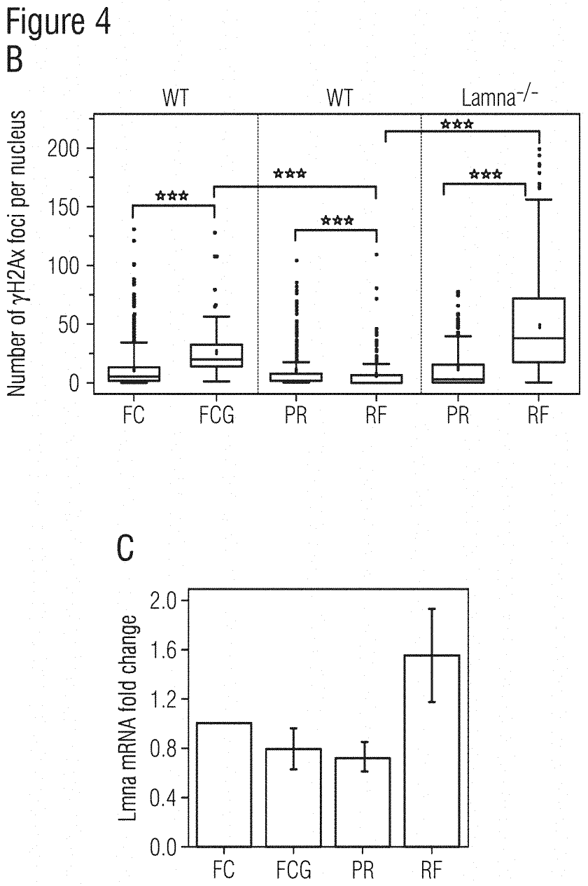

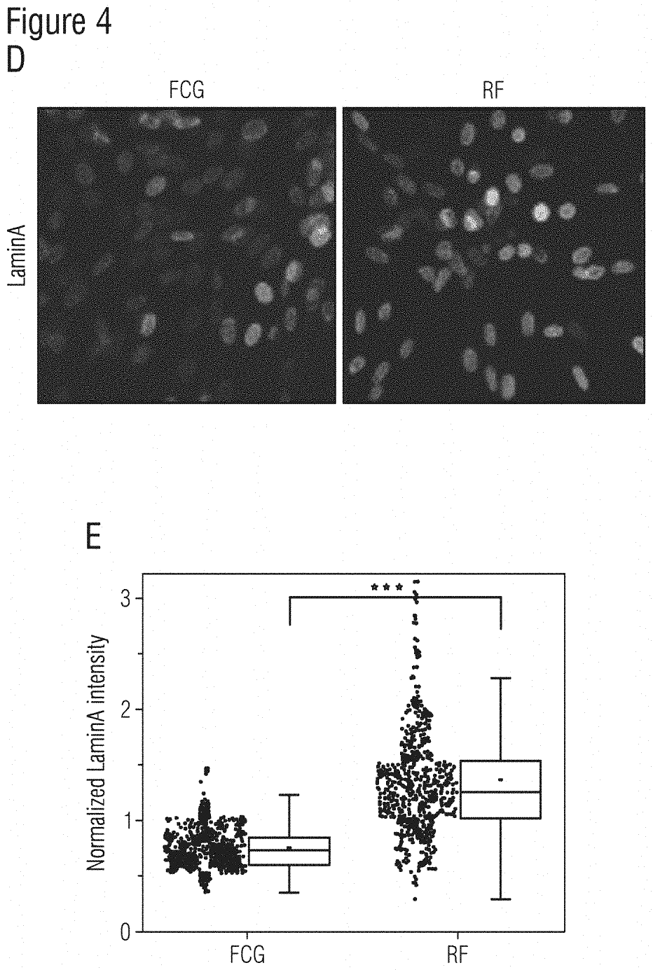

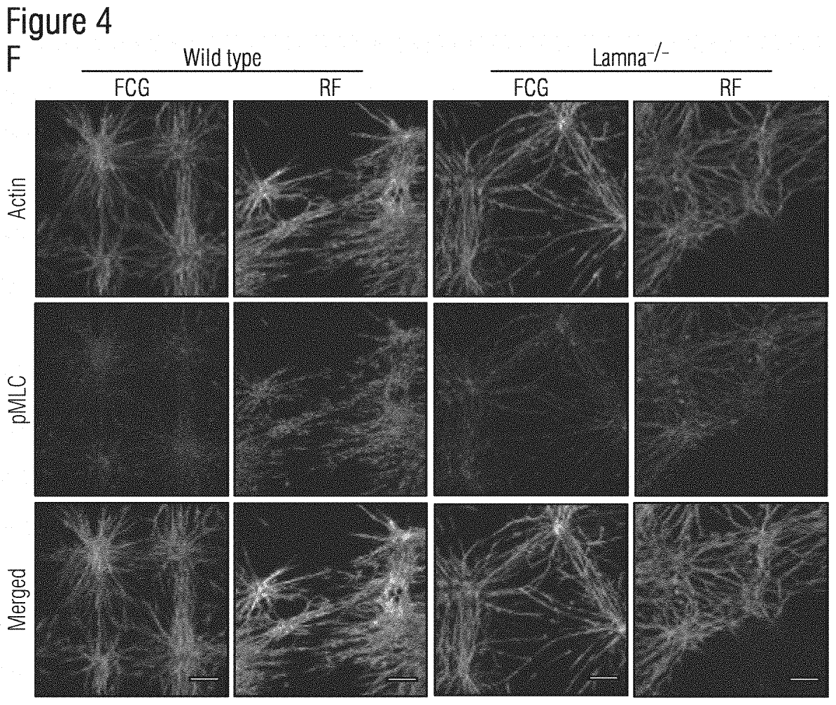

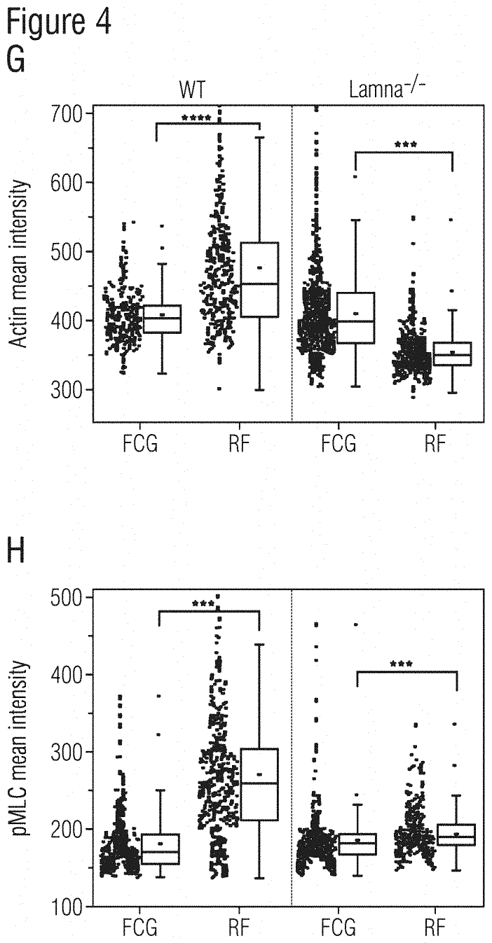

FIG. 4 recovering from Age-Associated Hallmarks DNA damage by Partial Reprogramming and LaminA dependency. (A) Representative gH2AX immunofluorescence micrographs of cell nuclei in different conditions: with and without gel, and in both wild type 3T3 (WT) and Lmna knockout 3T3 cell lines (Lmna−/−); scale bar: 10 μm. (B) Corresponding box plots of gH2AX foci per nucleus. n=549, 93, 522, 473, 323 and 545 for respective conditions. ***P<0.001; two-sided Student's t-test were used. (C) mRNA levels of Lmna in different conditions obtained by qRT-PCR. Normalized with respect to NIH3T3 clumps on patterns; Error bars represent SD. (D) Representative LaminA immunofluorescence micrographs of the nuclei in Fibroblasts' and 3T3 cells embedded in the collagen matrix. (E) Corresponding box plot for normalized nuclear LaminA intensities. n=633 and 554 for FCG and RF conditions respectively. ***P<0.001; two-sided Student's t-test were used. (F) Representative actin and pMLC immunofluorescence micrographs of Fibroblasts' and 3T3 cells embedded in collagen matrix in both WT and Lmna−/− conditions. In merged images, nucleus is in blue, scale bar: 50 μm. (G and H) Corresponding box plots for cellular mean intensity of actin and pMLC; n=400, 558, 1114, and 619 for the respective conditions. ***P<0.001; Two-sided Student's t-test were used ***P<0.001.

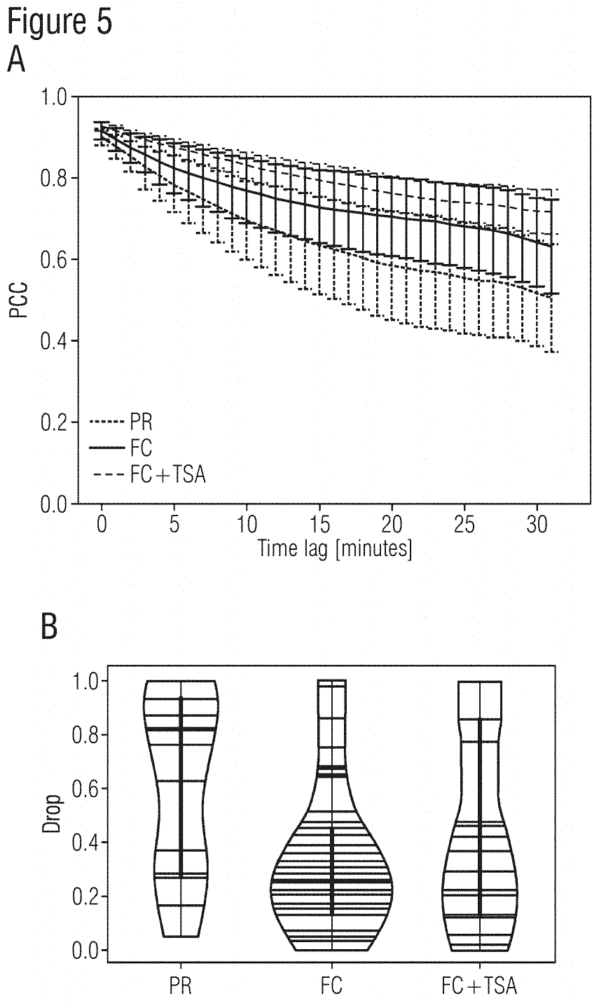

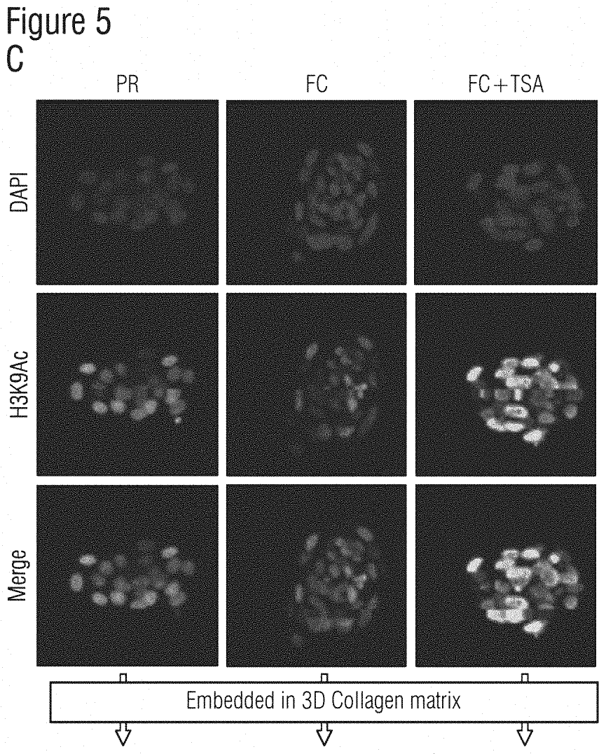

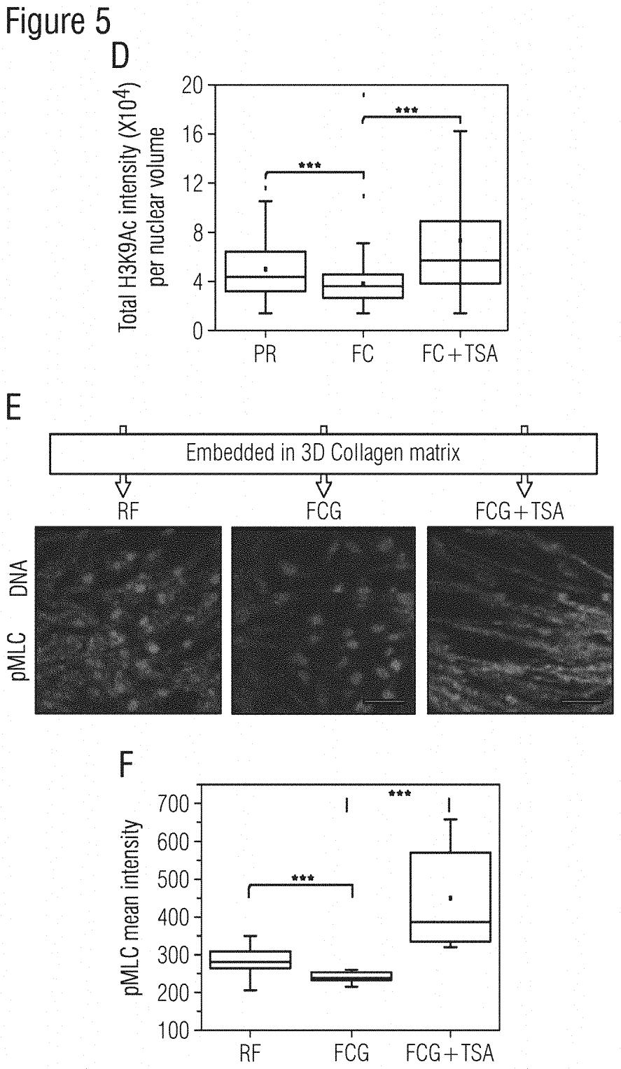

FIG. 5 chromatin Poised State in Partially Reprogrammed Cells. (A) Pearson correlation coefficient (PCC) curve of projected nuclear area as a function of time with mean and confidence interval of mean (as error bar). n=38, 138, 122 for PR, FC and FC+TSA conditions, respectively. (B) Drop rate obtained by fitting the nuclear area PCC curves of FIG. 5A indicating the rapid change of nuclear morphology and DNA organizations in PR and FC+TSA conditions. The procedure of analysis of FIGS. 5A and B are described in the method section. (C) Representative H3K9Ac immunofluorescence micrographs of the nuclei in Day 6, 3T3 clump, and 3T3 clump+TSA-treated cells without gel conditions, scale bar:10 μm. (D) Corresponding box plots of total H3K9Ac intensities per nuclear volume. n=383, 788 and 903 for PR, FC and FC+TSA, respectively. ***P<0.001; two-sided Student's t-test were used. (E) Representative pMLC immunofluorescence micrographs of the above-mentioned cell types embedded in collagen matrix for 2 days. Nucleus is labelled in red, scale bar: 50 μm. (F) Corresponding box plots of mean pMLC intensities in these three conditions. Number of field of view used for analysis, are n=23, 58, 15, and 15 for RF, FCG and FCG+TSA, respectively. ***P<0.001; two-sided Student's t-test were used. ***P<0.001; Student's t-test.

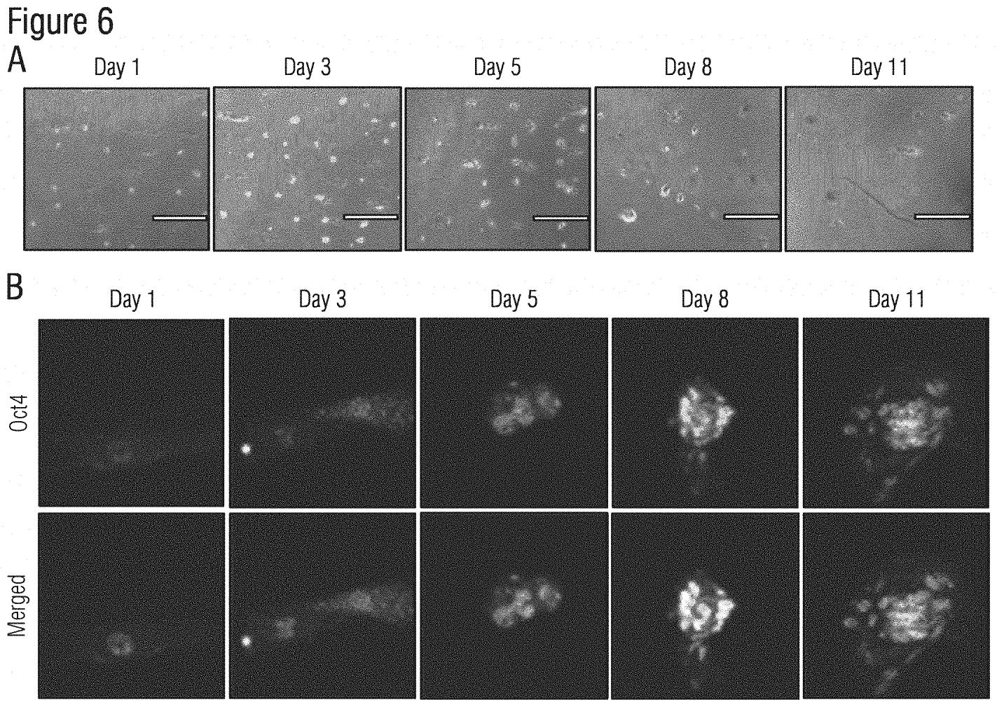

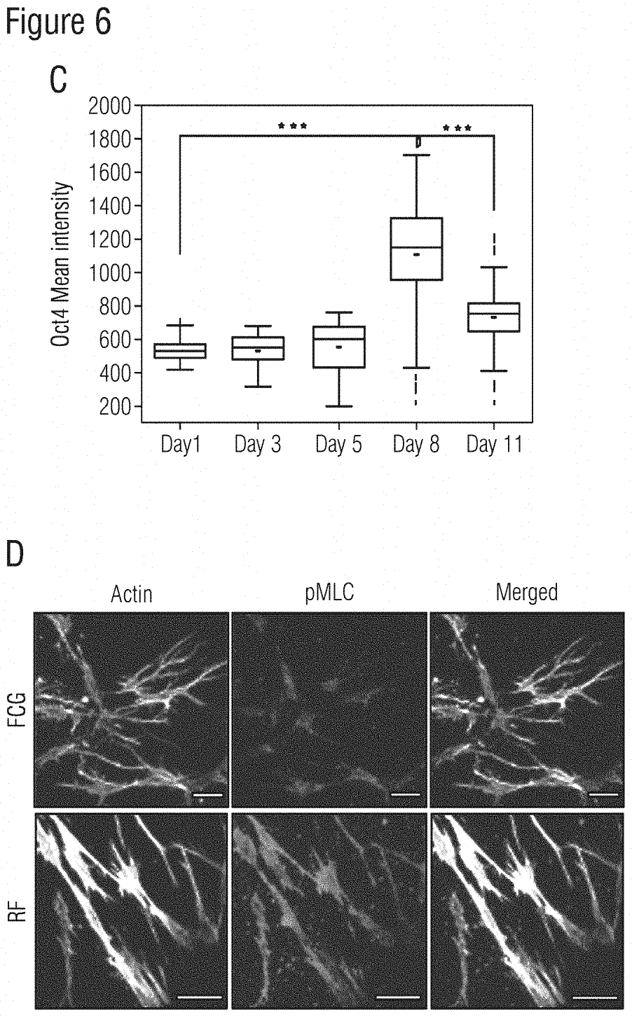

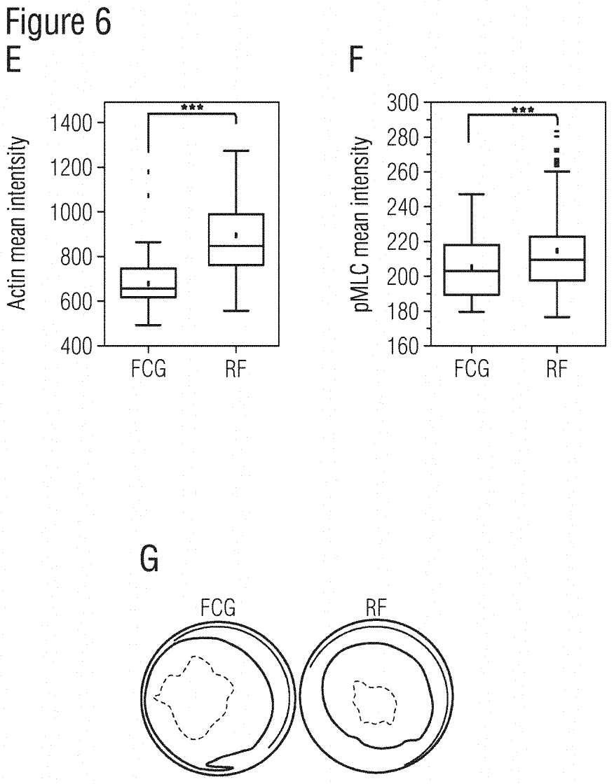

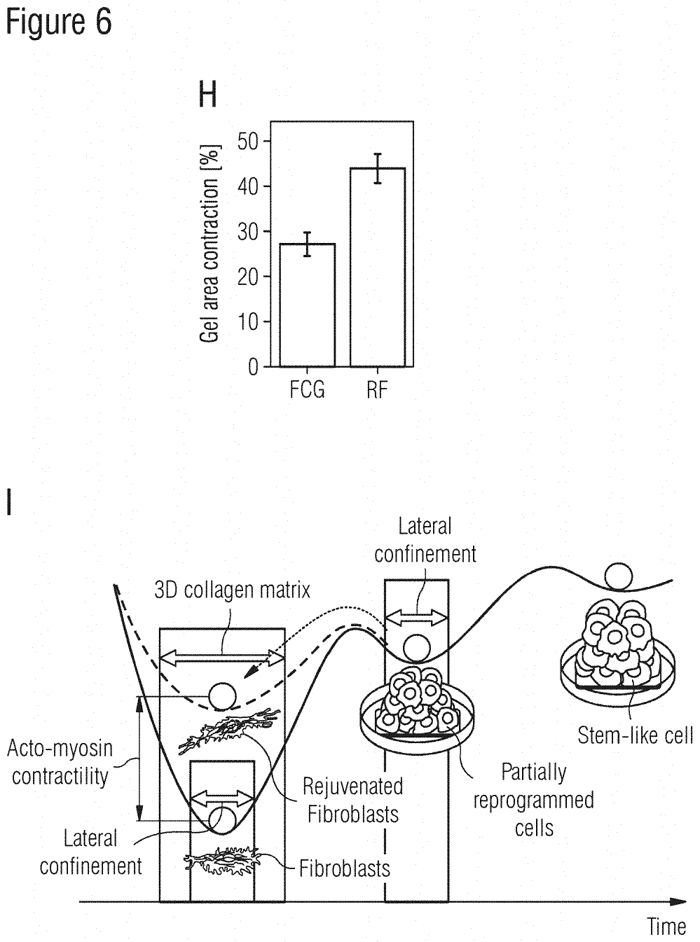

FIG. 6 rejuvenation of human aged fibroblasts. (A) Phase contrast images of human primary aged skin fibroblast (GM08401) cells cultured on micropatterns for up to 11 days, (scale bar: 500 μm); (B)

Representative micrograph of GM08401 spheroids at different days immunostained with Oct4, scale bar: 20 μm; (C) Nuclear Oct4 mean intensity plot of spheroids at different days. (D) Representative actin and pMLC immunofluorescence micrograph of RF and FCG of GM08401 embedded in 1 mg/ml collagen matrix; (E and F) Corresponding box plots for cellular mean intensity of actin and pMLC in FCG and RF conditions, respectively. ***P<0.001; two-sided Student's t-test were used (G) Representative images of collagen gel contraction by matrix-embedded with GM8401 RF and FCG. (H) Normalized gel area plot representing the gel contraction efficiencies of these two types of cells. (I) Schematic summary of the induction of reprogramming by laterally confined growth of cells and followed by redifferentiation into rejuvenated fibroblasts in 3D collagen matrix; and

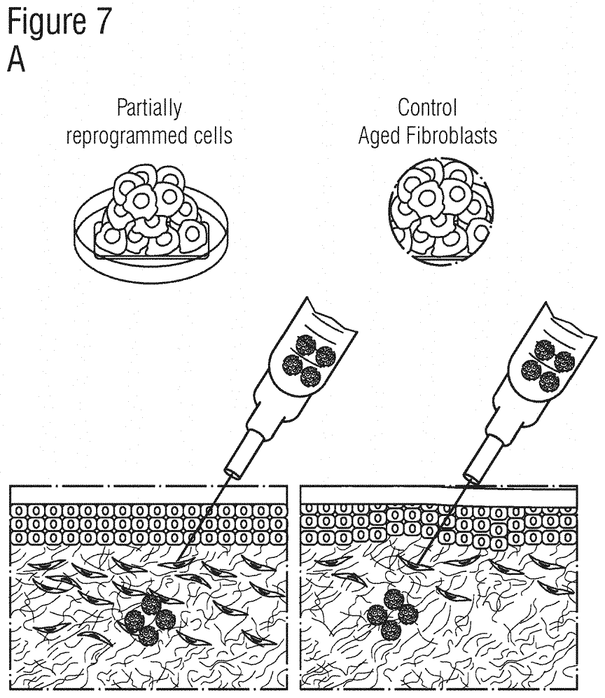

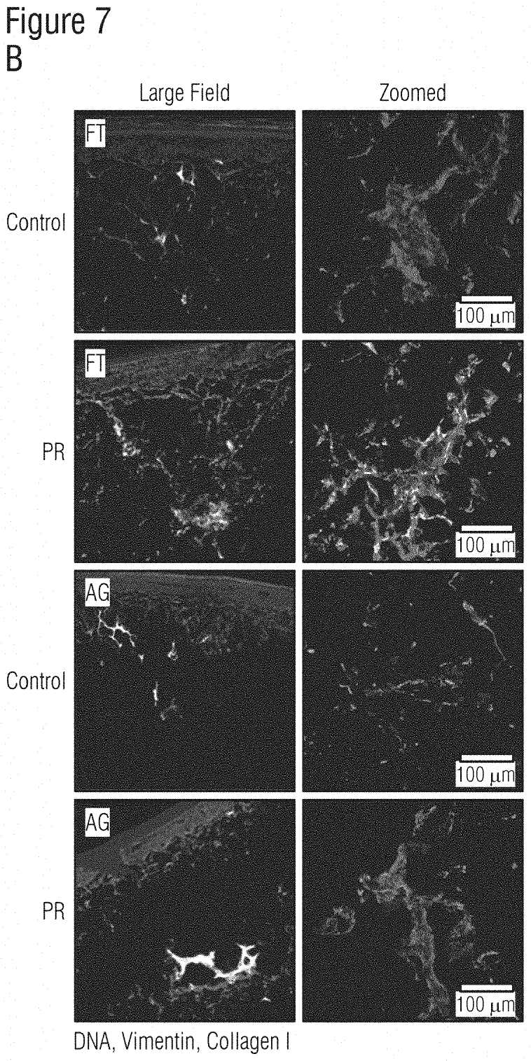

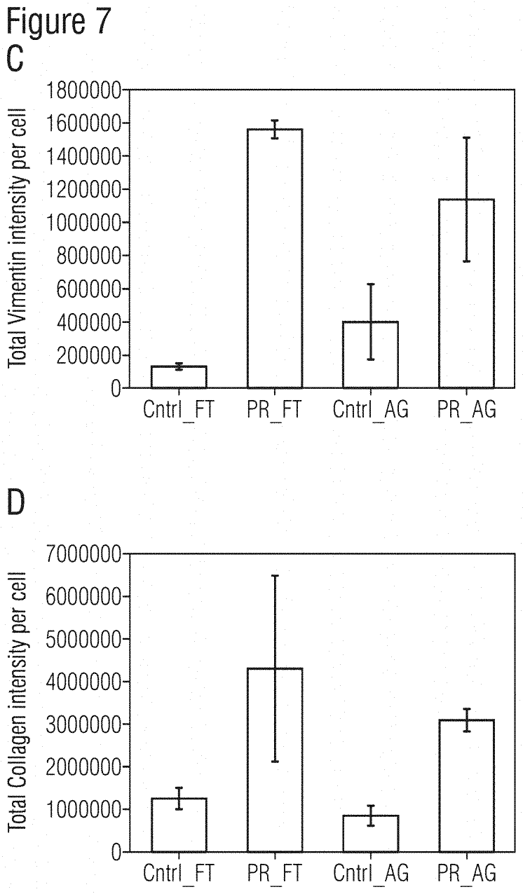

FIG. 7 rejuvenation of human aged fibroblasts in in vitro skin model. (A) Schematic representation of partially reprogrammed fibroblasts derived from human primary aged skin fibroblast (GM08401) injected into the in vitro full thickness and aged skin model (Phenion). The ability and efficiencies of the fibroblasts redifferentiation and matrix remodeling properties of these fibroblasts compared with control fibroblasts spheroids derived from aged fibroblasts after 10 days of redifferentiation, (B) Representative vimentin and collagen immunofluorescence micrographs of histological sections of the FT and AG in vitro skin model injected with either partially reprogrammed cells and control aged fibroblasts. (C and D) Corresponding quantification of total vimentin and collagen intensity in the injected cells and nearby matrix.

The present invention describes a unique method of fibroblast rejuvenation, which involves partial reprogramming of fibroblasts by growing them under lateral confinement, followed by their redifferentiation into fibroblast-like cells by embedding them in a 3D Collagen-I matrix. An appropriate 3D collagen matrix density for the redifferentiation process has been optimized. Here, fibroblast rejuvenation is demonstrated by revealing enhanced acto-myosin contractility, collagen remodeling, and matrix protein deposition in redifferentiated cells compared to parent fibroblasts. RNA sequencing reveals a shift in transcriptome from a fibroblastic to an intermediate reprogrammed state following lateral confinement, which shifts back to the fibroblastic transcriptome (enhanced expression of genes related to contractile cytoskeletal pathways) upon redifferentiation in the collagen matrix. Importantly, an amelioration of DNA damage is shown which is facilitated by an increase in laminA levels in the nucleus upon rejuvenation. In terms of changes to nuclear architecture, it is revealed that the comparatively open chromatin compaction state (chromatin poise state) induced by partial reprogramming is more likely to differentiate into contractile fibroblasts in response to ECM cues present in the 3D-collagen matrix than the parental fibroblasts. In summary, the present invention discloses that the mechanically-induced partial reprogramming approach described here overcomes the shortcomings of conventional rejuvenation methods, including generation of short-lived or oncogenic fibroblasts, and therefore has potential implications in the field of regenerative medicine.

### RESULTS

Redifferentiation of fibroblasts from partially reprogrammed spheroids depends on 3D collagen matrix density On mechanically-induced nuclear reprogramming in the absence of exogenous biochemical factors, it was found that mouse embryonic fibroblasts undergoing laterally confined growth for 6 days on micropatterns started to acquire partial stem cell-like gene expression. These 6 day-old spheroids were embedded in collagen gels and cultured for at least 2 days (FIG. 1A). Within few hours, cells originating from the spheroids progressively invaded the collagen matrix and migrated either individually as unicellular sprouts (single cells) or collectively as complex capillary-like structures (FIG. 1B). In addition to cell invasion, morphological modifications in the spheroid core itself were detected. While the spheroids initially appeared as a compact structure, subsequent cell migration induced spheroid expansion occurred and led to breaches in the spheroid core. Since cell/substratum adhesion is known to govern cell sprouting parameters in the 3D matrix, the influence of physical properties of the 3D matrix was next investigated, such as its porosity and stiffness (usually combined into a single parameter known as matrix density) on cell sprouting patterns. One compared cell sprouting in the matrix as a function of matrix density obtained through varying collagen concentration from 0.5 mg/ml to 2 mg/ml. By using AngioTools analysis to quantify cell sprouting from the spheroids on matrices of varying densities, it was found that average sprouting length in these cells showed a biphasic dependence on matrix density (FIGS. 1C and 1E). Similar to sprouting length, cell contractility, as measured by actin levels, also showed a similar biphasic trend depending on matrix density (FIG. 1F). Addition of cytoD (which depolymerizes actin) and blebbistatin (which inhibit myosin II contractility), led to a significant reduction in average sprouting length as well as a drop in collagen fiber stiffening.

One next investigated the effect of varying matrix density on cell proliferation rates, using an EdU incorporation assay to quantify DNA synthesis. By measuring the percentage of EdU (5-ethynyl-2′-deoxyuridine)-positive cells following cumulative incorporation (16 hrs), it was found that proliferation rates were significantly affected by matrix density (FIGS. 1D and G).

Furthermore, the effect of changing collagen density on both RF and FCG was compared. The control fibroblasts were embedded in three different collagen density i.e. 0.5, 1 and 2 mg/mL. It was observed that the sprouting efficiency and contractility (actin mean intensity) was increased with the increasing collagen densities. The proliferation of these FCG showed a biphasic trend with collagen densities similar to RF cells. Importantly, the sprouting length of FCG was relatively smaller than the RF condition in all three collagen densities suggesting that the RF cells could have higher cell-matrix contacts. The 1 mg/ml collagen concentration was therefore selected as the optimal matrix density in all subsequent studies. All these results suggest that an optimal mechanical state of the 3D collagen matrix result in the redifferentiation of partially reprogrammed cells into fibroblasts.

A shift in transcription profiles is accompanied by enhanced cytoskeletal gene expression: RNA-seq.

In order to characterize the gene expression profiles in redifferentiated fibroblasts (RF) and compare them with other control conditions, including partially reprogrammed cells (PR), fibroblasts grown in clumps (FC) and fibroblasts grown in clumps and embedded in collagen (FCG), RNA-seq experiments were performed. Thousands of genes, including key pluripotency markers Bmp4, Cdx2, Fgf4, Gdf3, Nanog, Nodal, Nt5e, Sall4 and Sox2, were solely upregulated in the PR cells (FIGS. 2A and B).

When the gene expression profiles in these four conditions were analysed, two drastically different cell states were revealed by principal component analysis (see method, FIG. 2C). PR cells shifted away from the parental fibroblast-like state (FC) to a stem-like state, as a result of the lateral confinement. Embedding these partially reprogrammed cells in the 3D collagen environment led their gene expression profiles to return to the parental 3T3 fibroblast-like state in RF cells. One observed a difference in gene expression profiles between cells undergoing reprogramming and original fibroblasts (both cultured on a 3D collagen matrix), which is supported by a Venn diagram showing the number of up-regulated genes in different comparisons (FIG. 2D). Based on these comparisons two groups of genes were identified, which were either selectively overexpressed (23 genes) or down regulated (53 genes) in RF compared to all the other conditions.

Further, of all the down regulated genes, one found a specific gene Follistatin (Fst) which is a common marker for aging and which was expressed at significantly lower levels in RF than in all other conditions (FIG. 2E). Such a significant decrease in Fst expression in RF indicates the rejuvenation of fibroblasts through the reprogramming process. In order to confirm the difference between Follistatin protein level in RF and FCG condition, Western blotting was performed. Consistent with the RNAseq result, the Follistatin protein level is lesser in the RF condition than FCG condition. Genes up-regulated in RF form a molecular interaction network, which is characterized by several connection nodes around proteins such as Rab25, Cdc42bpa, Rhoj and Iqgap1 that enhance cell migration and cell contractility (See Methods). The expression of selected genes regulating cell contractility was up-regulated in RF compared to FCG (FIG. 2F). Further, in agreement with the RNA-seq profile, the increase in mRNA levels of selected contractility-related genes was validated by qPCR assay (FIG. 2G). These experiments show that PR cells can be redifferentiated into a fibroblast-like (RF) state by embedding them into a 3D collagen matrix, and these cells are characterized by elevated expression of contractility- and rejuvenation-related genes.

### Redifferentiated Fibroblasts are Characterized by Enhanced Contractility and Matrix Remodeling

In order to characterize these RF in vitro, one compared the gel contraction abilities of cells derived from PR spheroids or from FC, both of which were equally embedded in the 3D collagen matrix. Representative images and quantitative analysis of gel area reduction revealed that the amount of collagen gel contraction by the RF was higher compared to FCG (FIG. 3A). Fibroblasts exhibit contractile actin bundles, and, therefore, one compared actin and phosphorylated myosin light chain (pMLC) global intensities in these two cell types embedded in the collagen matrix. In agreement with the RNA seq results, FIG. 3C-E clearly show that the RF exhibit enhanced actomyosin contractility compared to control fibroblasts (FCG). Fibroblasts are known to exert mechanical forces on the extracellular matrix surrounding them. Hence, one studied fibroblast-induced reorganization of the matrix by visualizing immunostained-collagen fibres and qualitatively evaluating the effect of different inhibitors on collagen fibre remodeling. Both RF and FCG embedded in fibrillary collagen matrix were able to remodel collagen fibrils into thicker bundles (FIG. 3F).

Remodeling of collagen fibrils into thick bundles was observed within the fibroblast-populated collagen gel. It was observed that collagen fibrils rearrange thicker around RF compared to control FCG samples. In addition, to check the expression level of collagen-cross linking molecules Lox in RF and FCG cells, the Lox mRNA level from the RNAseq data was plotted. RF cells shows higher Lox expression compared to FCG cells (FIG. 3D). This result suggests the enhanced remodeling properties of RF cells compared to FCG. However, collagen fibrils around RF exhibited very less or no remodeling in samples treated with 25 μM Y-27632 (a RhoA-kinase inhibitor) and 4 μM cytochalasin D (inhibitor of actin polymerization). Matrix assembly and remodeling is usually promoted by ECM glycoproteins that bind to cell surface receptors, such as fibronectin (FN) dimers binding to integrins. Fibroblasts deposit fibronectin to the matrix along the way of migration. Immunostaining experiments showed that both the fibroblasts migrated within the 3D collagen matrix, however, RF deposited more fibronectin along their migration trails compared to control FCG (FIG. 3G). In addition, these RF also expressed several other ECM-related genes including Lama1 (laminin, alpha 1), Fn1 (fibronectin 1), Col4a1 and Col1a1 at higher levels than in controls, as quantified by qPCR assay (FIG. 3H). Further, one performed western blotting analysis to confirm the difference between Follistatin protein level in RF and FCG condition. Consistent with the RNAseq result, the Follistatin protein level is lesser in the RF condition than FCG condition. Fibroblasts embedded in the 3D ECM are mechanically supported by the ECM and, in turn, exert forces onto the ECM through cell-ECM contacts.

A temporal quantitative measurement of the contractile forces exerted by these two fibroblast types was done by using 3D traction force microscopy (TFM) during fibroblast sprouting. A colour map based on the measurements indicated that RF exerted comparatively higher traction stress during the initial 12 hours of the sprouting phase (FIG. 3I). Vector arrows are indicated in the direction of force at each small window. During sprouting of cells from spheroids, the peak strain energy exerted by cells varied between spheroids, with a maximum energy of 450 pJ and 220 pJ exerted by RF and FCG, respectively (FIG. 3J).

Considering the non-linear properties of the collagen one also measured the traction force of FCG and RF cells seeded on a 2D fibronectin coated soft PDMS substrate (described in the methods section). Consistent with the 3D TFM results, it was observed that the RF cells showed higher 2D traction compared to the FCG cells. All together, these results support that augmented contractility and enhanced matrix remodeling are characteristics of the RF.

### Rejuvenation Through Redifferentiation of Partially Reprogrammed Fibroblasts Ameliorates Age-Associated Phenotypes

In order to investigate whether aging-associated phenotypes improve following rejuvenation, the level of DNA damage in these cells has been analyzed next. Interestingly, the number of foci containing histone gH2AX, a marker of nuclear DNA double-strand breaks associated with aging, were significantly reduced in RF compared to FCG (FIGS. 4A and B). Lateral confinement induces PR cells to accumulate significantly fewer gH2AX foci compared to FC. Sprouting of FC induced by constriction of pores in the 3D collagen matrix resulted in an increase in gH2AX foci, whereas, the change in the number of gH2AX foci in RF was insignificant compared to that in PR cells (FIGS. 4a and B).

Cell migration through constricting pores can lead to accumulation of DNA damage, which is dependent on its nuclear lamina levels. By using qPCR to quantify Lmna gene regulation, a decrease in Lmna mRNA levels was found in sprouted cells derived from FC compared to FCG (FIG. 4C). Interestingly, Lmna mRNA levels increased during redifferentiation in the RF. In agreement with the qPCR data, immunofluorescence data showed a significant increase in LaminA levels in the RF compared to FCG condition (FIG. 4D, 4E). In addition, an increase in the number of gH2AX foci in Lamna−/− RF suggests that higher LaminA levels in wild type RF may act to shield their nuclei from accumulating DNA damage during migration through constricted pores in the collagen matrix (FIGS. 4A and B). The nuclear lamina can regulate cellular contractility, and vice versa.

Therefore, the relationship between LaminA changes in rejuvenated cells and contractility has been investigated next. It was found that the actin level was significantly increased in RF compared to FCG, yet when lmna−/− cells were used, RF exhibited decreased actin compared to FCG (FIGS. 4F and G). However, an increase in pMLC levels in Lmna−/− RF was observed, although not as high as in wild-type RF (FIGS. 4F and H). Collectively, these results demonstrate that short-term, in vitro induction of fibroblast reprogramming through lateral confinement of 3T3 cells, followed by their subsequent redifferentiation can ameliorate phenotypes associated with physiological aging (e.g. accumulation of DNA damage and nuclear envelope defects) by increasing LaminA levels in their nuclei.

Chromatin poised states in partially reprogrammed cells The pluripotent genome is characterized by unique epigenetic features and a decondensed chromatin conformation. Therefore, it is hypothesized that rejuvenation of fibroblasts may be a result of the chromatin poised state in the PR cells. One first examined the nuclear dynamics in PR cells and FC and in FC treated with Tricostatin A (TSA), a specific inhibitor of histone deacetylase (HDAC). As expected, time-lapse laser scanning confocal microscopy of Hoechst 33342 stained nuclei showed an increase in nuclear dynamics in PR and TSA-treated FC, compared to control FC (FIGS. 5A and B). This also correlates with low levels of Lamin A in the PR nucleus compared to RF (FIG. 4C). Treatment with TSA increases the nuclear levels of H3K9ac, a marker of chromatin decondensation. In agreement with the nuclear dynamics results, immunofluorescence experiments clearly reveal a significant increase in H3K9ac levels in the PR and TSA-treated FC, compared to untreated FC (FIG. 5C, 5D). In order to explore levels of acto-myosin contractility, TSA-treated FC has been embedded in a 3D collagen matrix and the level of pMLC in sprouted cells has been assayed as a measure of contractility. Interestingly, a higher level of pMLC in the TSA-treated FCG compared to untreated FCG and RF has been found (FIG. 5E, 5F). These results suggest that during lateral confinement-induced reprogramming, cells undergo chromatin decondensation that may enhance the activity of target genes in response to matrix cues, promoting cellular processes essential for rejuvenation.

### Validation of Fibroblast Rejuvenation in Human Fibroblasts

In order to validate the rejuvenation results in the human fibroblast model the similar experimental approach to rejuvenate aged and young human fibroblasts was used. As an aged and young fibroblast model one used primary skin fibroblast obtained from aged donor (Age 75) (GM08401, Coriell Institute) and human foreskin fibroblasts cell line from newborn (BJ cells), respectively. GM08401 cells were grown on laterally confined condition on a specific fibronectin micropattern (area 9000 um² with aspect ratio 1:4) for 11 days until the spheroid formation (FIG. 6A). The partial reprogramming of the GM08401 was confirmed through the Oct4 immunostaining (FIG. 6B and C). Further one re-differentiated these partially reprogrammed GM08401 cells similarly by embedding them on a 1 mg/ml collagen matrix for 3 days until they sprout out in the collagen gel. Similarly, for control, one form the FC of aged cell and embedded in the collagen gel for FCG. In order to compare the contractility between the RF and FCG of aged cells, one measured the pMLC and actin level by immunofluorescence. Interestingly, one also observe the higher level of actin and pMLC in the RF cells compared to the FCG cells (FIG. 6D-F).

In addition, to show the gel contraction potential of these two type of fibroblasts, one performed the gel contraction assay similarly as described before. In agreement with the acto-myosin contractility, one also observed that gel with RF cells contract 44% whereas gel with FCG cells contract only 27% after 5 days of culture (FIGS. 6G and H). These results suggest that using similar approach, the aged cells can also be rejuvenated with higher acto-myosin contractility. To further validate the rejuvenation process in young human fibroblasts model, BJ cells were grown on laterally confined condition on a specific fibronectin micropattern for 10 days.

The partial reprogramming was confirmed through the increased level of alkaline phosphatase and Oct4 mRNA and protein expression. Further, one re-differentiated these partially reprogrammed BJ cells similarly by embedding them on a 1 mg/ml collagen matrix. These were then allowed to sprout and grow for 48 hours. The re-differentiated cells (RF) show higher contractility in terms of pMLC level as compared to the control BJ fibroblasts (FCG). Further, in agreement with the NIH3T3 cells, one also observed increased LaminA and decreased γH2AX levels in the re-differentiated BJ fibroblasts compared to control fibroblasts. In order to compare between the aged, rejuvenated fibroblasts derived from aged fibroblasts and young fibroblast, one used GM08401 (Age 75, Coriell Institute) and GM01652 (Age 11, Coriell Institute) as more appropriate aged and young human primary skin fibroblasts model, respectively. The rejuvenated fibroblasts (RF) were obtained from the aged primary skin fibroblast (GM08401, Age 75, Coriell Institute) similar to as mentioned previously in the manuscript. For control young and aged fibroblast condition, similarly one formed the FC of GM01652 and GM08401cells and embedded them in 1 mg/ml collagen matrix for respective FCGs.

A prominent characteristic of dermal fibroblasts in aged skin is reduced size, with decreased elongation and a more rounded, collapsed morphology. Whereas, young and healthy fibroblasts normally attach to the ECM strongly and thereby achieve stretched, elongated morphology. Importantly, one shows that the rejuvenated (RF) fibroblasts exhibit increased elongated morphology than its more rounded parental aged fibroblasts (GM08401) but similar to the control young fibroblasts (GM001652). The cell area analysis in 3D collagen matrix shows significant increased cell area upon rejuvenation of aged cells and the cell area of these RFs are similar to control young fibroblasts (FCG, GM01652).

Interestingly, one also observed the higher level of pMLC in the RF cells compared to the aged fibroblasts (GM08401) but similar to control young fibroblasts (GM01652). These results suggest that aged fibroblasts, upon rejuvenation, regain some of the characteristics of young fibroblasts (cell area and pMLC levels). Collectively, these results show similar type of rejuvenation characteristics of fibroblasts originated from either partially reprogrammed human (BJ) or mouse (NIH3T3) fibroblasts or aged fibroblasts (GM08401).

A schematic summary of the nuclear reprogramming processes induced by laterally confined growth of fibroblasts and their subsequent rejuvenation during redifferentiation within the 3D collagen matrix is shown in FIG. 6I.

FIG. 7 exemplarily shows the rejuvenation of human aged fibroblasts in an in-vitro skin model. (A) Schematic representation of partially reprogrammed fibroblasts derived from human primary aged skin fibroblast (GM08401) injected into the in vitro full thickness and aged skin model (Phenion). The ability and efficiencies of the fibroblasts redifferentiation and matrix remodeling properties of these fibroblasts compared with control fibroblasts spheroids derived from aged fibroblasts after 10 days of redifferentiation, (B) Representative vimentin and collagen immunofluorescence micrographs of histological sections of the FT and AG in vitro skin model injected with either partially reprogrammed cells and control aged fibroblasts. (FIGS. 7C and 7D) Corresponding quantification of total vimentin and collagen intensity in the injected cells and nearby matrix. Thus, the present invention paths a way of implanting partially reprogrammed fibroblasts directly into in-vitro skin model maintaining the cell viability and the rejuvenation efficiency. Further, the implanted partially reprogrammed cells are capable of expressing the selected fibroblast markers. As an important result, the rejuvenation of implanted cells in human in-vitro tissues is achieved and illustrates a possible way of implanting the partially reprogrammed fibroblast cells in human tissue.

### DISCUSSION

Somatic cell nuclear transfer (SCNT) and iPSCs have been used in cellular rejuvenation process in many studies, for example rejuvenation of aged fibroblasts, neurons, cardiac myocytes, T-cells, macrophages and skin cells. While all these methodologies have enormous applications, their clinical use is limited by disadvantages such as lower efficiency and increased risk of oncogenic transformations in cells due to genomic mutations acquired during the dedifferentiation process. Therefore, in recent years, several approaches, including environmental (heterochronic parabiosis), genetic (downregulation of NF-kB signaling) and pharmacological methods (mTOR inhibition by rapamycin can extend the life span of mice,) have been applied to rejuvenate cells without attaining complete dedifferentiation.

Despite the existence of several non-genetic approaches for dedifferentiation that involve the use of small molecules or cocktails of transcription factors, physical routes of reprogramming and their potential to overcome the above-mentioned limitations of dedifferentiation have not been clearly demonstrated. In addition, the rejuvenation of such physically dedifferentiated cells by their redifferentiation into more active cellular states have not been explored. In a recent study, it was shown that the laterally confined growth of fibroblasts on fibronectin micropatterns induces their reprogramming and confers on them ES-like characteristics. Along with the potential to restore stem cell-like properties, this mechanical mode of reprogramming also opens up avenue for potential implication in the field of rejuvenation.

In the present invention, one used these PR cells (generated by laterally confined growth of fibroblasts) with naive ES-like expression profiles and redifferentiated them into fibroblasts. This approach also highlights the advantage of decoupling rejuvenation from complete dedifferentiation. Redifferentiation of stem cells into a specific lineage can be augmented by the mechanical properties of the tissue microenvironment. Here one defines an optimal 3D mechanical microenvironment for the redifferentiation of partially reprogrammed spheroids, by controlling stiffness and pore size of the collagen-I matrix. The mechanical properties of the 3D collagen matrix, such as its stiffness, may act as regulatory checkpoints during the rejuvenation of fibroblasts on the matrix. These results suggest that optimal matrix stiffness and pore size (mimicking the architecture of the physiological tissue) induce efficient redifferentiation of these partially reprogrammed cells into fibroblasts.

Recent evidences from electron microscopy and electron spectroscopy imaging of chromatin structures indicate that undifferentiated ES cells and iPSCs exhibit an open chromatin state compared to differentiated cells. In agreement with these observations, fibroblasts that were partially reprogrammed under laterally confined growth conditions showed higher nuclear dynamics as well as enriched active histone marks (H3K9Ac), suggesting a more open chromatin structure compared to control fibroblasts. The open chromatin state is transcriptionally silent but poised for activation as its bivalent histone domains can be rapidly activated (through the loss of H3K27me3) when differentiation is induced.

During redifferentiation, PR cells migrate through a small mesh size of 2-4 um, enabling their open chromatin to be exposed to matrix signals. The cascade of collagen-I matrix dependent downstream signaling pathways in this highly open chromatin state may enable relatively increased transcription of their target genes leading to rejuvenation. Transcriptional analysis shows an upregulation of laminA, and other contractility and rejuvenation related markers in RF compared to control cells, suggesting that RF evolve from normal fibroblasts through reprogramming. In addition, treatment of fibroblasts with agents that promote chromatin decondensation, such as the HDAC inhibitor Trichostatin A, results in chromatin that is more poised for activation, as these cells showed increased contractility upon exposure to ECM-related cues.

The mechanical reprogramming of fibroblasts, followed by their redifferentiation into rejuvenated fibroblasts in an optimized 3D collagen matrix made these cells more contractile and more efficient at synthesizing matrix components including laminin, fibronectin, collagen-IV. Moreover, the rejuvenated fibroblasts obtained through this approach exhibited a decrease in DNA damage. The rejuvenated fibroblasts derived from this method precisely align into tissue architectures, suggesting its potential application as clinical implants in tissue engineering and regenerative medicine.

### METHODS

### Partial Reprogramming of Fibroblasts and Redifferentiation

NIH3T3 mouse embryonic fibroblasts were cultured on fibronectin micropatterns and grown under laterally confined conditions. Briefly, rectangular (aspect ratio 1:5) micropatterns measuring 1,800 μm² were created on uncoated Ibidi dishes (81151) by stamping of fibronectin (F1141, Sigma) coated PDMS micropillars fabricated by soft lithography. This was followed by surface passivation of the micropatterned dish with 0.2% pluronic acid (Sigma P2443) for 10 min. NIH3T3 cells were expanded in high-glucose DMEM (Gibco)+10% (vol/vol) FBS (Gibco) and 1% penicillin-streptomycin (Gibco).

For partial reprogramming, NIH3T3 cells were seeded on a fibronectin-micropatterned dish (rectangles spaced 150 μm apart) at a concentration of ˜7,000 cells per dish, to reach a density of one cell per fibronectin island. Single cells were grown in under laterally confined conditions for 6 days in the above-mentioned culture medium, with a fresh media replenishment on every alternate day unless otherwise stated.

In control 3T3 clump conditions (FC), similar spheroid size and cell density (compared to 6-day partially reprogrammed spheroids) was achieved by seeding NIH3T3 cells on differently spaced micropattern dishes (500 μm) at a concentration of ˜80,000 cells per dish and growing them overnight. For redifferentiation, spheroids or cells obtained from trypsinized spheroid were embedded in 3D rat tail Collagen-I gel of varying concentration (0.5-2 mg/mL) according to the manufacturer's protocol (Thermofisher). In such a 3D collagen matrix, cells were cultured for 48 h in the above mentioned medium for most of the rejuvenation assays unless otherwise stated.

In order to partially reprogram human foreskin fibroblasts (BJ cells), cells were grown on laterally confined condition similar to NIH3T3 cells on a specific fibronectin micropattern (area 3364 um², AR 1:4)) for 10days in high-glucose DMEM (Gibco) +10% (vol/vol) FBS (Gibco) and 1% penicillin-streptomycin (Gibco). Similar to re-differentiation of NIH3T3 cells, these partially reprogrammed BJ cells were further re-differentiated by embedding them on a 1 mg/ml collagen matrix. The aged and young human primary skin fibroblasts were obtained from aged donor (Age 75) (GM08401, Coriell Institute) and young donor (Age 11), (GM01652, Coriell Institute), respectively. GM08401 cells were cultured and grown on laterally confined condition on a specific fibronectin micropattern (area 9000 um² with aspect ratio 1:4) in a 1:1 high-glucose DMEM (Gibco) and MEM (Gibco) media supplemented with 15% (vol/vol) heat inactivated FBS (Gibco) and 1% penicillin-streptomycin (Gibco). For respective young and aged fibroblasts, FCs were obtained on fibronectin micropattern (area 9000 um² with aspect ratio 1:4) and followed by embedding them in 1 mg/ml collagen matrix for their respective FCGs.

### Cell Proliferation Assay

The percentage of cells (cultured in Collagen-I gel for 24 hours) in the S phase was evaluated by using an in situ cell-proliferation kit (Click-iT™ EdU Alexa Fluor™ 555 Imaging Kit, Thermofisher scientific) that quantified the incorporation of 5-ethynyl-2′-deoxyuridine (EdU) into cellular DNA. As per the manufacturer's instructions, cells in 3D Collagen-I matrix were allowed to incorporate 10 uM EdU for 16 hours. After EdU incubation, cells were fixed with 4% paraformaldehyde and permeabilized with 0.5% Triton for 20 minutes. Following this, cells were incubated with 0.5 mL of Click-iT® reaction cocktail for 30 to 35 minutes at room temperature and then washed with PBS. Cell nuclei were counterstained with DAPI.

### RNA-Seq Sample Preparation and Analysis

Total RNA was isolated from cells grown on patterns for varying times by using the RNeasy Plus Micro Kit (Qiagen). Cells grown in 3D Collagen-I gel were treated with collagenase for 15 minutes prior to RNA isolation. The preparation of the mRNA library (Illumina Stranded) and sequencing on a HiSeq 2000 platform was performed at the Genome Institute Singapore. In summary, we had four conditions: FC (3T3 clumps grown overnight on micropatterns without gel), FCG (3T3 clumps grown in Collagen-I gel for 48 h), PR (partially reprogrammed spheroids without gel) and RF (6-day samples grown in Collagen-I gel for 48 h); each condition had three biological replicates and four technical replicates (run on four different lanes). Reads were aligned to Mus musculus GRCm38.p6 soft-masked genomic DNA (with GenBank Assembly ID GCA_000001635.8, downloaded from Ensembl) using the tophat sequence alignment tool. The annotation file (GTF format) used for tophat sequence alignment was downloaded from Ensembl (for GRCm38.p6 assembly) (31). Default parameters were used in Tophat (v2.1.1) (32). After alignment, four technical replicates for each biological sample (accepted hits.bam files from tophat output) were combined together for downstream analysis. Cufflinks (v2.2.1) software was used to assemble the transcripts and obtain the number of reads for each transcript(33). The number of reads for transcripts from the same gene were summed to get the count number (reads per million, RPM). Count numbers for all expressed genes were used in differential expression analysis using DESeq2 (Version 1.20.0) (34). Differentially expressed genes have adjusted p values (Benjamini-Hochberg) below a 0.1 false discovery rate (p value thresholds used in other analyses are described in respective figure legends).

Quantitative Real-Time PCR (qRT-PCR).

To investigate selected gene expressions in the four conditions mentioned above, qRT-PCR was performed. Using iScript cDNA synthesis kit (Bio-rad), cDNA was synthesized from total RNA that was isolated as mentioned previously. Real-Time PCR detection was performed using SsoFast qPCR kit (Bio-Rad) for 40 cycles in a Bio-Rad CFX96. The relative fold changes in the gene levels were obtained from qRT-PCR data, by using ΔΔCt methods with respect to GAPDH levels.

### Collagen-I Contraction Assay

To initiate the gel contraction assay, an equal number of cells obtained from trypsinized 6 day spheroids or clumps were mixed with Collagen-I solution and cast in new, uncoated Ibidi dishes (81151) and cultured for 5 days. As a measure of fibroblast contractility, Collagen-I gel contraction by the fibroblasts was performed by measuring the fractional decrease in gel area with time.

### Traction Force Microscopy

Quantitative measurement of the force exerted by cells embedded within a collagen matrix was measured by 3D traction force microscopy. Briefly, cells in PR spheroids or FC were fluorescently labelled with Cytotracker red (ThermoFisher) before they were embedded within the collagen matrix. Fluorescent beads of 4 μm size were mixed with 1 mg/ml Collagen-I gel composition at a concentration of 60,000-80,000 beads per 400 μl of gel solution. Subsequently, fluorescently-labelled spheroids or clumps were mixed with collagen solution and cast in uncoated ibidi dishes for polymerization at 37° C. in a CO₂ incubator. Once cells started to migrate within 4 hrs of incubation, the bead and cell displacements were tracked for 18 hrs under a confocal microscope, at an interval of 30 minutes. Traction forces were analyzed using fourier transform traction cytometry. Particle displacement rates were calculated using particle-tracking velocimetry, based on algorithms. Particle displacements were interpolated into a regularized grid corresponding to the approximate substrate displacement. Particle-tracking errors were eliminated using the filtering procedure. From the displacement field, the traction maps were quantified using matlab. For 2D traction force measurement, we prepared the soft PDMS substrate for traction force as described by Das et. al.

Briefly, one prepared the ultra-soft polydimethylsiloxane (PDMS) substrate by mixing base (Sylgard 184, Dow Corning Corp. Mid-land, Mich., USA) to cross-linker in 65:1 ratio (w/w). Fluoro-sphere of diameter 46±6 nm (Molecular probes, ThermoFisher) were added to the PDMS mixture, followed by careful stirring for at least 15 mins. The PDMS-bead mixture was spread on to ibidi glass bottom dishes. The coated dishes were then kept at 25° C. for a period slightly more than one hour. Subsequently, the PDMS coated dishes were incubated at 50° C. for 4 h for desired cross-linking. These were then treated with oxygen plasma and coated with fibronectin 10 ug/ml for 2 hr at 37° C. RF and FCG were isolated from the collagen gel by partial treatment of collagenase and trypsinization. The single cell of RF and FCG were seeded on the TFM substrate at a low density and incubated overnight. Calculation of the displacement field from the fluorescent images of the bead-embedded substrate with and without cells were performed. From the displacement field, the traction maps were quantified using matlab.

### Immunostaining

Cells embedded in Collagen-I gel were fixed with 4% Paraformaldehyde (Sigma) in PBS buffer (pH 7.4) for 25 min, followed by washing with PBS+100 mM glycine buffer (5 min×3). Cells were permeabilized using 0.5% Triton (Sigma-Aldrich) in PBS for 20 min, followed by washing with PBS-glycine buffer (5 min×3). After that, cells were blocked with 10% goat serum (ThermoFisher Scientific) in IF wash buffer (PBS+0.2% Triton+0.2% tween 20) for 3 h at room temperature. Next, cells were incubated overnight with different primary antibodies diluted in blocking buffer, followed by washing with IF wash buffer (15 min×3). Cells were then incubated with corresponding fluorescent-labeled secondary antibodies diluted in 5% goat serum in IF wash buffer for 3 h at room temperature. Cell nuclei were stained with NucBlue Live Ready Probes (Molecular Probes; Thermo Fisher Scientific) in PBS for 10 min at room temperature, and filamentous actin was labeled using phalloidin Alexa Fluor 488 or 568 (1:100; Molecular Probes; Thermo Fisher Scientific) for 45 min.

### Image Acquisition and Analysis

Fluorescent images of 3D spheroids and cells embedded in 3D Collagen-I gel were acquired by using Nikon AIR laser scanning confocal microscope (Nikon Instruments Inc, Japan), at either 20× magnification (Plan Apo 20× ELWD, NA 0.8) or 63× magnification (1.25 NA oil objective) with identical acquisition settings. In the Z dimension, each spheroid and 3D Collagen-I gel was scanned up to a depth of 50 μm, with a step size of 1 to 5 μm. Confocal images of either 512×512 or 1024×1024 pixels were obtained with an XY optical resolution of 0.42 μm or 0.21 μm, respectively. Time lapse imaging was done in confocal mode for up to 60 min and 18 h with 60 s and 30 m time intervals, respectively. Bright-field images were acquired using the EVOS FL Cell Imaging System (Thermo Fisher Scientific). For gel contraction assay, images of collagen gel at different days of cell growth were acquired with a mobile camera at a fixed magnification. The fluorescence intensity was measured for each protein in its respective channel and the number of gH2Ax foci per nucleus was determined using custom-written code in Fiji (NI) MATLAB (Mathworks) and IMARIS8. The sprouting length of each spheroid embedded in 3D collagen matrix was measured from the large field fluorescent micrograph of actin using AngioTools software.

Nuclear dynamics were analyzed from the decorrelation of the nuclear images in time as described previously(37). Time-lapse live-imaging of nucleus stained with Hoechst 33342 (ThermoFisher Scientific) was done in confocal mode with time intervals of one minute for up to 32 minutes in PR, FC and FC+TSA conditions. One Pearson correlation coefficient (PCC) value was calculated from two lists of pixel intensity of the same nucleus captured in different time points with a certain time lag. For each cell, one PCC curve was drawn which connecting all PCCs (as y) with the increasing time lags (as x) as represented in dim color in FIG. 5A. The mean PCC curves for all cells in each condition, were drawn in bright color in FIG. 5A. The mean PCC curves in each condition were fitted by equation y=(1−α)+α exp(−t/τ)−η, where y refers to PCC value, t is time lags, fitting parameter α is drop rate, τ is time constant, η is noise.

### Statistical Analysis

All data are expressed as mean±SD or ±SEM as noted in figure legends. For box plots, box limit represents the 25-75 percentile and whiskers 1.5× interquartile range. Each experiment was repeated at least three times. We evaluated statistical significance of mean with the student's unpaired two-tailed t test, performed between sample of interest and corresponding control. *P<0.05; **P<0.01; ***P<0.001.

1-9. (canceled)

10. A method for fibroblast rejuvenation by mechanical reprogramming and redifferentiation, the method comprising the following steps:
a) laterally confined growing of fibroblasts on micro-patterned substrates in order to induce a generation of stem cell-like spheroids, being partially reprogrammed spheroids; and
b) embedding the partially reprogrammed spheroids in three-dimensional (3D) matrices of varying densities, thereby mimicking different 3D tissue constraints.

11. The method according to claim 10, which comprises embedding the partially reprogrammed spheroids in collagen-I matrices.

12. The method according to claim 10, wherein the micro-patterned substrates are fibronectin micropatterns.

13. The method according to claim 12, wherein the fibronectin micropatterns are rectangular micropatterns having an aspect ratio of approximately 1:5 and measuring in a range of 400 to 3,000 μm², created on uncoated cell culture dishes by stamping fibronectin-coated polydimethylsiloxane (PDMS) micropillars formed by soft lithography.

14. The method according to claim 13, wherein the cell culture dishes are Ibidi® dishes.

15. The method according to claim 13, wherein the rectangular micropatterns measure approximately 1,800 μm².

16. The method according to claim 10, which comprises surface passivating the micropatterned substrate with pluronic acid and expanding fibroblast cells in high-glucose DMEM (Dulbecco's Modified Eagle Medium) and FBS (fetal bovine serum) and penicillin-streptomycin.

17. The method according to claim 10, which comprises partially reprogramming by seeding the fibroblast cells on a fibronectin-micropatterned dish at a density of one cell per fibronectin island, and growing single cells under laterally confined conditions for a predetermined amount of time in the culture medium.

18. The method according to claim 17, wherein the micropatterned dish has rectangles spaced apart by approximately 150 μm and a cell concentration of 2,000 to 20,000 cells per dish, and the predetermined amount of time is approximately two days.

19. The method according to claim 17, which comprises replenishing the culture medium with fresh media every other day.

20. The method according to claim 17, which comprises setting the cell concentration at approximately 7,000 cells per dish.

21. The method according to claim 10, which comprises partially reprograming human fibroblasts (BJ cells), by growing the human fibroblast cells on laterally confined condition on a specific fibronectin micropattern in high-glucose DMEM (Dulbecco's Modified Eagle Medium) and FBS (fetal bovine serum) and penicillin-streptomycin, and further re-differentiating the partially reprogrammed human fibroblast cells by embedding the cells on a collagen matrix.

22. The method according to claim 21, wherein the specific fibronectin micropattern has an area in a range from 1,000 to 10,000 μm².

23. The method according to claim 22, wherein the specific fibronectin micropattern has an area of 3,364 μm² at an aspect ratio of 1:4.

24. The method according to claim 21, which comprises growing the human fibroblast cells for a period of two days.

25. The method according to claim 10, which comprises using micro-patterned substrates for partially reprogramming fibroblasts with high efficiency.

26. The method according to claim 10, which comprises executing the step of partial reprogramming with patient specific old fibroblasts.

27. The method according to claim 10, which comprises establishing a 3D gel protocol to encapsulate reprogrammed old fibroblasts for their rejuvenation.

28. The method according to claim 10, which comprises characterizing patient specific rejuvenated fibroblasts for potential applications.
